(19) United States
(12) Patent Application Publication
(10) Pub. No.: US 2024/0307465 A1
(43) Pub. Date: Sep. 19, 2024

(54) METHODS OF PREVENTING, DELAYING OR AMELIORATING ATOPIC DISEASES

(71) Applicant: Johnson & Johnson Consumer Inc., Skillman, NJ (US)

(72) Inventors: Kamyar Farahi, Blue Bell, PA (US); Richard A. Insel, Rochester, NY (US); Dolores Heidi Needleman, Doylestown, PA (US)

(21) Appl. No.: 18/038,519

(22) PCT Filed: Nov. 22, 2021
(86) PCT No.: PCT/IB2021/060830

§ 371 Date: May 24, 2023

Related U.S. Application Data
(60) Provisional application No. 63/117,562, filed on Nov. 24, 2020.

(51) Int. Cl. A61K 35/745; A61K 9/00; A61K 35/00; A61K 35/20; A61P 17/00

(52) U.S. Cl. CPC A61K 35/745; A61K 9/0095; A61K 35/20; A61P 17/00; A61K 2035/115

(57) ABSTRACT

Provided are compositions methods of preventing, delaying or ameliorating atopic dermatitis in a breastfed infant having an increased risk of developing an atopic disease, the method comprising administering a composition comprising administering a Bifidobacterium to the breastfed infant.

### FIELD

The present invention generally relates to methods of preventing, delaying, or ameliorating an atopic disease, such as atopic dermatitis, and particularly to methods comprising administering a composition comprising an effective amount of a Bifidobacterium to the breastfed infant.

### BACKGROUND

Atopic diseases are a class of diseases in which the immune system develops immunoglobulins to common environmental allergens which are generally considered to be harmless. One example of an atopic disease is atopic dermatitis (“AD”). AD, also known as atopic eczema, is a chronic inflammatory allergic skin disorder that frequently develops in childhood. AD is characterized by eczematous lesions, i.e., erythematous patches with eruption, blistering, and crusting, that, when chronic, become scaling with fissures and lichenification, with intense pruritus.

As the most prevalent pediatric allergic disease, AD affects up to 30% of children. Although the incidence of AD peaks in infancy, disease commonly persists and/or recurs into adulthood. There are numerous topical treatments (lotions, ointments, etc.), but none are able to completely cure eczema. Accordingly, there is an ongoing need for treatments which are effective to prevent, delay and/or ameliorate the onset of atopic dermatitis (as well as other atopic diseases) in infants.

### SUMMARY OF THE INVENTION

Accordingly, one aspect of the invention pertains to a method of preventing, delaying or ameliorating atopic dermatitis in a breastfed infant. In one or more embodiments, the method comprises administering a composition comprising an effective amount of a Bifidobacterium selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof to the breastfed infant.

In some embodiments, wherein the Bifidobacterium is selected from the group consisting of B. longum, B. breve, B. kashiwanohense and combinations thereof. In one or more embodiments, wherein the Bifidobacterium is a B. longum subspecies selected from the group consisting of longum, suis, infantis, and combinations thereof. In some embodiments, wherein the Bifidobacterium is B. infantis. In one or more embodiments, wherein the Bifidobacterium is mixed into breastmilk prior to administering the Bifidobacterium to the breastfed infant. In some embodiments, wherein the Bifidobacterium is mixed into infant formula prior to administering the Bifidobacterium to the breastfed infant. In one or more embodiments, wherein the Bifidobacterium is mixed with about 3 to about 5 mL of breastmilk, infant formula or water prior to administering the Bifidobacterium to the breastfed infant. In some embodiments, wherein the breastfed infant is greater than 50% breastfed. In one or more embodiments, wherein the breastfed infant is exclusively breastfed. In some embodiments, wherein the Bifidobacterium is in powder form mixed with lactose. In one or more embodiments, wherein about 5 to about billion CFU of the Bifidobacterium are administered to the breastfed infant. In some embodiments, wherein the B. infantis is administered once daily. In one or more embodiments, wherein the B. infantis is first administered within the first 2 weeks of life. In some embodiments, wherein the B. infantis is administered before the first 12 weeks of life. In one or more embodiments, wherein the Bifidobacterium is first administered within the first 2 weeks and until the 12^{th} week of life. In some embodiments, wherein the breastfed infant has an increased risk of developing an atopic disease. In one or more embodiments, where in the Bifidobacterium comprises the strain EVC001.

Another aspect pertains to a method of preventing, delaying, or ameliorating atopic dermatitis in a breastfed infant having at least one first-degree relative with history of an atopic disease, the method comprising: administering once daily an effective amount of B. infantis mixed with breastmilk to the breastfed infant, wherein the breastfed infant is at least 90% breastfed, and wherein the B. infantis is first administered within first 2 weeks of life.

In some embodiments, wherein the B. infantis comprises strain EVC001. In one or more embodiments, wherein the B. infantis is mixed with about 3 to about 5 mL of breastmilk or infant formula prior to administering the B. infantis to the breastfed infant. In some embodiments, wherein the breastfed infant is exclusively breastfed. In one or more embodiments, wherein the B. infantis is in powder form mixed with lactose. In some embodiments, wherein 8 billion CFU of B. infantis are administered to the breastfed infant. In one or more embodiments, wherein the B. infantis is administered once daily until the 12^{th} week of life.

Another aspect pertains to a method of preventing, delaying or ameliorating an atopic disease selected from the group consisting of food allergy, allergic rhinitis, asthma and combinations thereof in a breastfed infant, the method comprising administering a composition comprising an effective amount of a Bifidobacterium.

In some embodiments, wherein the Bifidobacterium is selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof to the breastfed infant. In one or more embodiments, wherein the Bifidobacterium is selected from the group consisting of B. longum, B. breve, B. kashiwanohense and combinations thereof. In some embodiments, wherein the Bifidobacterium is a B. longum subspecies selected from the group consisting of longum, suis, infantis, and combinations thereof. In one or more embodiments, wherein the Bifidobacterium is B. infantis. In some embodiments, wherein the Bifidobacterium is mixed into breastmilk prior to administering the Bifidobacterium to the breastfed infant. In one or more embodiments, wherein the Bifidobacterium is mixed into infant formula prior to administering the Bifidobacterium to the breastfed infant. In some embodiments, wherein the Bifidobacterium is mixed with about 3 to about 5 mL of breastmilk, infant formula, or water prior to administering the Bifidobacterium to the breastfed infant. In one or more embodiments, wherein the breastfed infant is greater than 50% breastfed. In some embodiments, wherein the breastfed infant is exclusively breastfed. In one or more embodiments, wherein the Bifidobacterium is in powder form mixed with lactose. In some embodiments, wherein about 5 to about 15 billion CFU of the Bifidobacterium are administered to the breastfed infant. In one or more embodiments, wherein the B. infantis is administered once daily. In some embodiments, wherein the B. infantis is first administered within the first 2 weeks of life. In one or more embodiments, wherein the B. infantis is administered before the first 12 weeks of life. In some embodiments, wherein the Bifidobacterium is first administered within the first 2 weeks and until the 12^{th} week of life. In one or more embodiments, wherein the breastfed infant has an increased risk of developing an atopic disease. In some embodiments, where in the Bifidobacterium comprises the strain EVC001.

Another aspect pertains to a method of improving infantile colic, infant sleep or infant anthropometrics in a breastfed infant, the method comprising administering a composition comprising an effective amount of a Bifidobacterium.

In one or more embodiments, wherein the Bifidobacterium is selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatil, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof to the breastfed infant. In some embodiments, wherein the Bifidobacterium is selected from the group consisting of B. longum, B. breve, B. kashiwanohense and combinations thereof. In one or more embodiments, wherein the Bifidobacterium is a B. longum subspecies selected from the group consisting of longum, suis, infantis, and combinations thereof. In some embodiments, wherein the Bifidobacterium is B. infantis. In one or more embodiments, wherein the Bifidobacterium is mixed into breastmilk prior to administering the Bifidobacterium to the breastfed infant. In some embodiments, wherein the Bifidobacterium is mixed into infant formula prior to administering the Bifidobacterium to the breastfed infant. In one or more embodiments, wherein the Bifidobacterium is mixed with about 3 to about 5 mL of breastmilk, infant formula, or water prior to administering the Bifidobacterium to the breastfed infant. In some embodiments, wherein the breastfed infant is greater than 50% breastfed. In one or more embodiments, wherein the breastfed infant is exclusively breastfed. In some embodiments, wherein the Bifidobacterium is in powder form mixed with lactose. In one or more embodiments, wherein about 5 to about 15 billion CFU of the Bifidobacterium are administered to the breastfed infant. In some embodiments, wherein the B. infantis is administered once daily. In one or more embodiments, wherein the B. infantis is first administered within the first 2 weeks of life. In some embodiments, wherein the B. infantis is administered before the first 12 weeks of life. In one or more embodiments, wherein the Bifidobacterium is first administered within the first 2 weeks and until the 12^{th} week of life. In some embodiments, wherein the breastfed infant has an increased risk of developing an atopic disease. In one or more embodiments, where in the Bifidobacterium comprises the strain EVCO001.

These and other features and advantages of the present invention will be readily apparent from the following detailed description of the invention.

### DETAILED DESCRIPTION OF THE INVENTION

As used herein, the term “effective amount” means an amount sufficient to induce the desired effect. The term “safe amount” means an amount that is low enough to avoid serious side effects. The safe and/or effective amount of the compound, extract, or composition will vary with, e.g., the age, health and environmental exposure of the end user, the duration and nature of the treatment, the specific extract, ingredient, or composition employed, the particular pharmaceutically-acceptable carrier utilized, and like factors.

As used herein, “essentially free” or “substantially free” of an ingredient means containing less than 0.1 weight percent, or less than 0.01 weight percent, or none of an ingredient.

One aspect of the invention pertains to a method of preventing, delaying or ameliorating an atopic disease in a breastfed infant having an increased risk of developing an atopic disease, the method comprising administering a composition comprising an effective amount of a Bifidobacterium to the breastfed infant. Another aspect of the invention pertains to use of a Bifidobacterium in preventing, delaying or ameliorating an atopic disease in a breastfed infant having an increased risk of developing an atopic disease. The atopic disease may be selected from the group consisting of food allergy, allergic rhinitis, asthma, and combinations thereof. The atopic disease may be atopic dermatitis.

AD, which arises from a combination of defective epidermal skin barrier function, T-cell activation, and dysbiosis of skin commensal microbes, may precede the onset of other atopic disease, e.g., food allergy, asthma, and allergic rhinitis, in the so-called “atopic march.” Owing to transcutaneous allergic sensitization through eczematous skin, approximately one-third of children with AD develop food allergy. In fact, a unique AD endotype associated with food allergy has been identified and is characterized by altered terminal epidermal differentiation with changes in collagen expression, T-helper 2 (Th2) immune transcripts, poor skin barrier function, and predisposition to cutaneous Staphylococcus aureus colonization and infection.

Another aspect of the invention pertains to a method of improving infantile colic, infant sleep or anthropometrics in a breastfed infant having an increased risk of developing the atopic disease, the method comprising administering a composition comprising an effective amount of a Bifidobacterium. Another aspect of the invention pertains to use of a Bifidobacterium in improving infantile colic, infant sleep disturbances or anthropometrics in a breastfed infant having an increased risk of developing the atopic disease.

Improvements or amelioration with respect to any of the above conditions may be measured by known methods in the art. For example, improvement in the severity of atopic dermatitis may be measured using the Eczema Area and Severity Index (EASI), which is further defined below. Improvement in infantile colic may be indicated by meeting fewer of the Rome IV criteria (defined below) or by a reduction in crying or fussing events. Improvement in infant sleep may be measured using a BISQ-R score (further discussed below) or by a reduction in one or more sleep pattern events, such as sleep onset latency, number and duration of night wakings, longest stretch of sleep, total night sleep, etc.) Improvements or amelioration may be with respect to the condition in an infant without being treated with the Bifidobacterium.

As used herein, the term “increased risk of developing an atopic disease” refers to an infant having one first-degree relative having a history of atopic disease (i.e., biological parent or full sibling with mother-reported, physician-diagnosed AD, allergic rhinitis, or asthma). The infants may be born either vaginally or via C-section.

It has been surprisingly discovered that the incidence of AD, other atopic disease, infantile colic, infant sleep, or infant anthropometrics can be affected, even in infants who are breastfed. The greatest risk factor for AD is a family history of atopic disease, with a 50% increased risk of disease if one parent has atopic disease and a 300% increased risk if one parent has AD. Genetic susceptibility to AD arises from multiple genetic loci, including semi-dominant loss-of-function mutations of the skin intermediate filament filaggrin (FLG) gene, which impairs skin barrier function, and the Th2 cytokine cluster locus on chromosome 5q31.1. Early-life determinants play a role in the development of atopic diseases including AD. The gut microbiome, owing to its pivotal role in developing and regulating a healthy immune system versus the dysregulated responses associated with allergic disease, heavily influences the development of childhood-onset AD and allergy. Association studies of the neonatal and infant gut microbiome and AD have shown an altered gut microbiome with decreased diversity and increased concentrations of fecal Bacteroidaceae and Enterobacteriaceae in the neonatal period. Conversely, a lower relative abundance of Bifidobacteriaceae and Lactobacillaceae is associated with the development of AD.

Results of studies conducted in the art thus far have been inconsistent, with probiotics reducing the incidence and severity of AD in some, but not all, studies. The pathophysiology of AD is complex and multifactorial, involving elements of skin barrier dysfunction (in some cases, mediated loss of function mutations in the FLG gene), alterations in cell-mediated immune responses, IgE-mediated hypersensitivity, and environmental factors. The gut microbiome communicates with the skin as one of the main regulators in the gut-skin axis, e.g., the gut microbiome can influence both innate and adaptive immunity through the production of secreted factors or metabolites that can enter circulation and thus elicit systemic effects. This link plays an important role in maintaining skin homeostasis by supporting epithelial-differentiation and immunoregulation. Conversely, the gut microbiome participates in the pathophysiology of inflammatory disorders of the skin including psoriasis and AD.

Contributing to the variability of findings are heterogeneity of trial design, study population, and diagnostic criteria and use of different probiotics that, in all cases, were never demonstrated to colonize the infant gut or to affect infant immune training. While not wishing to be bound to any particular theory, it is thought that predominant colonization of the gut with B. infantis or similar Bifidobacterium early in life will promote immunoregulation and decrease the risk of AD onset in the first year of life in at-risk infants.

The gut of an infant is sterile before birth, and the microbiome that subsequently develops is heavily affected by how the infant is born and by whether it is breastfed or not. It is generally thought that infants born via caesarean section (C-section) develop different gut microbiome profiles from vaginally-delivered infants because they do not pass through the birth canal. Instead, C-section infants are more likely to be exposed to bacteria of their environment. Additionally, breastmilk itself has probiotic properties and contains oligosaccharides which help to stimulate Bifidobacteriaceae and Lactobacillaceae growth. Thus, it would be expected that breastfeeding an infant would help to restore the microbiome profile of an infant to an ideal profile, regardless of how the infant was born.

### Bifidobacterium

Bifidobacterium is a genus of gram-positive, anaerobic bacteria, which reside in the gastrointestinal, vaginal, and oral tracts of mammals, including humans. The suitable Bifidobacterium may be those having at least one human milk oligosaccharides (HMO) gene cluster. The Bifidobacterium may be one that is similar to B. infantis. The Bifidobacterium may be selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof. The Bifidobacterium may be selected from the group consisting of B. longum, B. breve, B. kashiwanohense and combinations thereof. The Bifidobacterium may be B. longum. The Bifidobacterium may be a subspecies of B. longum selected from the group consisting of longum, suis and infantis.

As used herein, the term “Bifidobacterium infantis” or “B. infantis” is meant to refer to the subspecies of Bifidobacterium longum subsp. infantis. The B. infantis may comprise the strain EVC001. B. infantis can be isolated and cultured using methods known in the art.

The B. infantis may be co-administered with one or more other probiotics (i.e., other bacteria which are intended to have health benefits). The other probiotics may be strains selected from Lactobacillus, Lacticaseibacillus and Bifidobacterium genera. Examples of Bifidobacterium species include other strains of B. infantis, B. Longum (subspecies other than B. infantis), B. Breve, B. catenulatum, B. adolescentis, B. animalis, B. gallicum, B. lactis, B. pseudocatenulatum and B. Bifidum. Examples of Lactobacillus strains include L. paracasei, L. acidophilus, L. johnsonii, L. delbrueckii, L. crispatus, L. gasser, L. zeae, and combinations thereof. Examples of Lacticaseibacillus include L. casei, L. rhamnosus and combinations thereof. Other probiotics include Lactiplantibacillus plantarum, Limosilactobacillus fermentum and Ligilactobacillus salivarius. Alternatively, the Bifidobacterium may be administered without any other probiotics. That is, the Bifidobacterium may be formulated to be essentially free of any other probiotics.

The infant's gut microbiome profile can be tested and monitored to determine colonization by Bifidobacterium using methods known in the art. Stool samples may be used in such methods.

### Composition

The Bifidobacterium may be formulated into a composition which is easy to use and allows for consistent dosing. The fermentation product from Bifidobacterium production may be concentrated and freeze dried to provide a concentrated powder. The composition may contain about 1 million, 500 million, 1 billion, 2 billion, 3 billion, 4 billion, 5 billion, 6 billion, 7 billion, 8 billion, 9 billion, 10 billion or 12 billion to about 8 billion, 9 billion, 10 billion, 20 billion, 30 billion, 40 billion, 50 billion, 60 billion, 70 billion, 80 billion, 90 billion, 100 billion, 200 billion, 250 billion or 500 billion colony forming units (CFU) of Bifidobacterium per gram dry weight.

The Bifidobacterium may also be formulated with an oligosaccharide. As used herein, the term “oligosaccharide” refers to a saccharide polymer containing 2 to 20, 2 to 10, 3 to 20 or 3 to 10 monosaccharide units. The oligosaccharide may be those found in a mammalian milk (e.g., human, or bovine). The oligosaccharide may be synthesized.

The composition containing the Bifidobacterium may also contain an auxiliary component. Such auxiliary component are those commonly used in the art and may be selected from metabolites, flow agents or combinations thereof. Examples of flow agents include starch, silicon dioxide, cellulose, sodium bicarbonate, calcium silicate and the like. The auxiliary component may also be a milk protein or constituent. The auxiliary component may comprise lactose. That is, in such an example, the Bifidobacterium is in powder form mixed with lactose.

The final form of the composition can be any known in the art. As described above, the Bifidobacterium may be in dried form (e.g., spray-dried or freeze-dried) as a powder. Said powder may be dosed as a packet, sachet, tablet, foodstuff, capsule, lozenge, tablet, suspension, dry form, etc.

A Bifidobacterium product which is suitable in accordance with one or more embodiments of the invention is Evivo® probiotic available from Evolve BioSystems (Davis, CA), which are packaged in a sachet containing 8 billion CFU of B. infantis (EVC001) co-formulated with lactose.

### Administration of Bifidobacterium

As used herein, the term “administering” refers to providing a given dose of Bifidobacterium to infants as part of their feeding (i.e., it is used as a food supplement). The Bifidobacterium may be mixed with any medium that can be consumed by the infant, including breast milk, infant formula, water or food prior to administering the Bifidobacterium to the infant. The Bifidobacterium may be mixed into breastmilk prior to administering the Bifidobacterium to the breastfed infant. Alternatively, the Bifidobacterium may be mixed into infant formula prior to administering the Bifidobacterium to the breastfed infant. The Bifidobacterium is mixed with enough infant formula or breastmilk so that the infant is able to completely incorporate the Bifidobacterium and so that the infant is still likely and able to consume the entire dose of Bifidobacterium. Thus, the Bifidobacterium may be mixed with about 3 to about 5 mL of breastmilk or infant formula prior to administering the Bifidobacterium to the breastfed infant. The Bifidobacterium composition may be mixed by any suitable means, including simply stirring (or any other suitable means to obtain a mixture) the composition with the medium (e.g., infant formula, breast milk, water) in a bowl. The composition mixed with infant formula or breastmilk may then be fed to the infant by any suitable means. Suitable means of feeding to the infant include use of a feeding syringe, spoon, or bottle. The Bifidobacterium may be administered prior to feeding the infant when the infant is more likely to be hungry, which is thought to increase the likelihood of the infant consuming the entirety of the dose.

The dose and dosing frequency may be selected as desired. For example, the Bifidobacterium may be administered once daily. In such an example, the dose once daily may contain from about 5-15 billion or about 8 billion CFU. Splitting the total desired dose into smaller doses is also contemplated. Examples could include smaller doses several times throughout the day (e.g., 2, 3, 4 or 5 times per day).

The total dose given per day may range from about 1 million, 500 million, 1 billion, 2 billion, 3 billion, 4 billion, 5 billion, 6 billion, 7 billion, 8 billion, 9 billion, 10 billion or 12 billion to about 8 billion, 9 billion, 10 billion, 20 billion, 30 billion, 40 billion, 50 billion, 60 billion, 70 billion, 80 billion, 90 billion, 100 billion, 200 billion, 250 billion or 500 billion colony forming units (CFU) of the Bifidobacterium. The total dose given per day may range from about 5 to about 15 billion CFU, or be about 8 billion CFU. Such total dose values may be given in one dose.

The Bifidobacterium may be administered beginning on the 1^{st}, 2^{nd}, 3^{rd}, 4^{th}, 5^{th}, 6^{th} day or first week of life, or beginning within the first 2, 3, 4, 5, 6, 7, 8, 9, 10, 11 or 12 weeks of life, or beginning with the first 3, 4, 5, 6, 7, 8, 9, 10, 11 or 12 months of life. As used herein the term “of life” means after birth. Once started, the Bifidobacterium may continue to be administered until the 4^{th}, 5^{th}, 6^{th}, 7^{th}, 8^{th}, 9^{th}, 10^{th}, 11^{th}, 12^{th} week of life, or until the 3rd, 4^{th}, 5^{th}, 6^{th}, 7^{th}, 8^{th}, 9^{th}, 10^{th}, 11^{th} or 12^{th} month of life. The Bifidobacterium may be first administered within the first 2 weeks of life. The Bifidobacterium may be first administered within the first 2 weeks of life and until the 12^{th} week of life.

The infants may be breastfed infants. As used herein, the term “breastfed” means that the infant derives at least some of its sustenance from human breastmilk. The infant may either nurse or the breastmilk may be expressed (e.g., pumped or hand-expressed) and given to the infant. The breastfed infant may be at least about 50, 60, 75, 80, 90% or 95% breastfed. The remainder of the infant's sustenance may be derived from infant formula or other food. Alternatively, the breastfed infant may be exclusively breastfed. As used herein, the term “exclusively breastfed” means that the infant does not receive infant formula, except that small amounts of infant formula may be used for the sole purpose to mix with the Bifidobacterium and administer to the infant. Any caloric contribution from other sources during the first 3 months of life, including medicines, the Bifidobacterium composition, or any medium used to deliver the Bifidobacterium, etc. is considered negligible.

While the foregoing description represent exemplary embodiments of the present invention, it will be understood that various additions, modifications, and substitutions may be made herein without departing from the spirit and scope of the present invention. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description. It will be appreciated that in the claims, the term “comprises/comprising” does not exclude the presence of other elements or steps. In addition, singular references do not exclude a plurality. The terms “a”, “an”, “first”, “second”, etc., do not preclude a plurality.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

All percentages, parts and ratios are based upon the total weight of the composition of the present invention, unless otherwise specified. All such weights as they pertain to the listed ingredients are based on the level of the particular ingredient described and, therefore, do not include carriers or by-products that may be included in commercially available materials, unless otherwise specified.

### Prophetic Example

This clinical trial will investigate the clinical and immunological effects of B. infantis (strain EVC001; Evolve BioSystems, Inc., Davis, CA) supplementation beginning within 14 days of birth and continuing for 12 weeks. Randomized, placebo-controlled experimental studies aim to decrease or eliminate known confounding effects from factors other than the study intervention.

This type of study design is thus considered to be rigorous because it is able to test causal relationships between the study intervention (in this case, the Study Supplement) and study endpoints.

### Objectives

The primary objective of this study is to assess the effect of B. infantis (EVC001) versus placebo supplementation, in healthy breastfed infants at risk of developing AD, on the cumulative incidence of physician-diagnosed AD during the first year of life. The secondary objective of this study is to assess the effect of B. infantis (EVC001) versus placebo supplementation, in healthy breastfed infants at risk of developing AD, on:
The proportion of infants with adverse events (AEs) in order to evaluate safety and tolerability
The cumulative incidence of AD at additional timepoints
The time to onset of AD
B. infantis colonization of the infant gut
AD severity for subjects with AD

Additional objectives of this study are:
1) To assess the effect of B. infantis (EVC001) versus placebo supplementation, in healthy breastfed infants at risk of developing AD, on:
   Incidences of atopic disease other than AD (food allergy, allergic rhinitis, asthma)
   Allergic sensitization
   Gut and skin microbiome
   Skin immunological biomarker profile
   Incidence of infantile colic
   Infant sleep
   Infant anthropometrics
   Relationship between baseline maternal gut microbiome and the development of the infant gut microbiome
2) To characterize and compare microbiome and immunological profiles between infants who do not develop AD through 1 year and infants who do in the B. infantis (EVC001) supplementation group and in the placebo supplementation group.
3) To determine the immune responder phenotype in the B. infantis (EVC001) supplementation group and in the placebo supplementation group.
4) To identify the subject genetic profile linked to benefit from the supplement intervention.

### Overview

This study is a randomized, double-blind, placebo-controlled, 2-arm, parallel-group (Groups 1 and 2) study. The study will enroll approximately 286 infants with at least one first-degree relative having a history of atopic disease (i.e., biological parent or full sibling with mother-reported, physician-diagnosed AD, allergic rhinitis, or asthma) who are currently breastfed, with maternal intent to maintain exclusive breastfeeding for at least 12 weeks (approximately 3 months). Each infant will participate in the study under the supervision of his/her biological mother (“Caregiver”).

Infant eligibility evaluations will be conducted within the first 14 days of life. Eligible infants will be enrolled and randomized evenly (1:1) to one of two groups: placebo (Group 1) or B. infantis (EVC001) (Group 2). The randomization will be stratified by the number of the infants' first-degree relatives (one versus more than one) having a history of relevant atopic disease (as defined above). All baseline assessments will be conducted prior to the first administration of the assigned Study Supplement.

Administration of the assigned Study Supplement will commence on Day 0 and continue for 12 weeks. Caregiver will be instructed to make their best effort to maintain exclusive breastfeeding for at least this 12-week period, and they will be encouraged to continue breastfeeding for as long as possible during the first year of life. Following the 12-week Supplementation Period, infants will be followed through Week 104 (approximately 2 years) via scheduled and unscheduled visits.

A sub-study is planned with a subset of the main study population (approximately 80-100 subjects) to evaluate the possible relationships between the intestinal microbiome, frequency, and function of specific immune cells in the peripheral circulation, the circulating cytokine profile, and the development of AD.

### Subject Selection and Enrollment

The eligibility criteria are designed to select subjects for whom protocol procedures are considered appropriate. Infant eligibility will be assessed within the first 14 days of the newborn's life. The initial verification of eligibility may be conducted by a non-medically qualified individual.

The inclusion criteria of the infant includes the following:
1) Male or female newborn ≤14 days old at the time of study enrollment (Day 0).
2) Healthy term infant.
3) Has at least one first degree relative (i.e. biological parent or full sibling) with a history of atopic disease (i.e., mother-reported, physician-diagnosed AD, allergic rhinitis, or asthma).
4) Breastfeeding established at the time of study enrollment (Day 0), with maternal intent to maintain exclusive breastfeeding for ≥12 weeks.

The exclusion criteria of the infant includes the following:
1) Preterm delivery (<36 weeks [252 days] gestational age).
2) Admission to the neonatal unit for issues other than establishment of normal feeding.
3) Evidence of a baseline illness/condition (e.g. abnormal birth weight) or significant risk of developing an illness/condition (based on review of maternal/pregnancy information) that would, in the opinion of the PI or designee, introduce a significant safety concern if the infant were enrolled in the study or otherwise preclude study participation.
4) Significant birth defect/complication that would, in the opinion of the PI or designee, create a safety concern or otherwise confound the study (e.g., abdominal wall defects, congenital heart disease).
5) Severe widespread skin condition (e.g., collodion).
6) Medical condition (infant) or maternal medication/supplement use (e.g., daily or routine antibiotics or systemic antifungals) that, in the opinion of the PI or designee, may significantly alter the gut or skin microbiome.
7) Has consumed a prebiotic or (a) Bifidobacterium longum-containing probiotic supplement/milk/formula prior to enrollment (Day 0).
8) Has consumed >100 mL of formula per day within the 48 hours prior to enrollment (Day 0).
9) Medical condition (infant) or maternal surgery/injury/condition that would preclude breastfeeding.
10) Known infant sensitivity to, or intolerance of, soya or dairy protein consumption.
11) Maternal infection with human immunodeficiency virus, tuberculosis, hepatitis C, or hepatitis B.
12) Caregiver condition that, in the opinion of the PI or designee, would not allow the Caregiver and/or infant to comply with the study protocol requirements. 13) Twin or multiple births.

During the study, the infant's Caregiver will be directed to make their best effort to ensure the infant is exclusively fed breastmilk through at least Week 12, and encouraged to continue breastfeeding for as long as possible during the first year of life. The Caregiver will also administer the assigned Study Supplement to the infant once daily for 12 weeks according to provided instructions and training. The Caregiver will be instructed to avoid routine infant ingestion of probiotics for the first 12 weeks of the study (or during the breastfeeding period, if longer), unless specifically prescribed by an HCP, e.g., to prevent or treat antibiotic-associated diarrhea or treat gastroenteritis. The Caregiver will also be directed to ensure that the infant does not ingest any prebiotics or any Bifidobacterium-containing probiotic supplement/milk/formula during the first 24 weeks of the study.

### Sample Size, Randomization/Study Supplement Allocation and Blinding

Approximately 200-400 subjects will be randomly assigned in a 1:1 ratio to receive B. infantis (EVC001) or placebo. Enrolled infants will be randomized evenly (1:1) to the active or placebo supplementation arm according to a randomization schedule. Infants will be stratified by the number of first-degree relatives (one versus more than one) having a history of atopic. The study will be double-blinded, so that the Caregivers and the PI/designees do not know the Study Supplement assignment.

### Identity and Use of Study Supplements

The following Study Supplements will be provided, as shown in Table 1 below:

TABLE 1
Study Supplements

| Identification | Product type |
|---|---|
| B. infantis (EVC001) | Active supplement |
| Matching lactose | Placebo supplement |
| placebo | |

The active supplement is B. infantis supplement Evivo® probiotic powder available from Evolve BioSystems, Inc. in sachets and having ingredients: purified lactose, B. longum subsp. Infantis EVC001 per dose. Each sachet contains 625 mg of the probiotic powder and contains 8 billion CFU of B. infantis (EVC001). The placebo sachets contain 625 mg of lactose.

Infants will ingest the contents of a single-serving sachet of B. infantis (EVC001) or matching placebo Study Supplement once daily for 12 weeks. At the time of supplementation, the contents of a single-serving sachet will be mixed with approximately 3-5 mL of expressed or pumped breastmilk (or infant formula, if needed) in a provided reservoir. Using the provided syringe, the mixture will be dispensed into the side of the infant's mouth to ensure the infant ingests the entire dose.

### Study Duration, Procedures and Evaluation Schedule

Infant eligibility evaluations will be conducted within the first 14 days of the infant's life. Following enrollment and randomization (Day 0), the Study Supplement will be administered daily from Day 0 through Week 12. Caregivers will be instructed to make their best effort to maintain exclusive breastfeeding for ≥12 weeks. Following the 12-week Supplementation Period, study subjects will be followed for an additional 92 weeks to complete this 104-week (2-year) study.

### Assessment Tools and Additional Study Procedure Details

### Medical, Family, Medication, and Supplement History Questionnaire:

At Screening, Study Personnel will interview each infant's Caregiver to complete the Medical, Family, Medication, and Supplement History Questionnaire to document the infant's medical history and medication/supplement history, including exposures during/from pregnancy, birth, and breastfeeding. The questionnaire will also capture mother-reported information about the infant's first degree relatives having a history of atopic disease, for example current or prior history of AD (including age of onset and details of diagnostic testing), allergic rhinitis/hay fever (including identification of allergens), asthma, food reaction/allergy (such as type of food and reaction; details of any other formal allergy testing), other skin conditions, or an immune-mediated disease.

### Diagnosis of AD:

Infants with possible cases of AD identified by Study Personnel will be assessed by a trained physician for evaluation and diagnosis. Briefly, AD will be diagnosed if three of the following four criteria are met: 1) pruritus, 2) typical morphology and distribution (facial and extensor involvement), 3) chronic or chronically relapsing dermatitis, 4) personal or family history of atopic disease (Rajka G, Langeland T. Grading of the severity of atopic dermatitis. Acta Derm Venereol Suppl (Stockh) 1989; 144:13-4.; Gånemo A, Svensson Å, Svedman C, Grönberg BM, Johansson A C, Wahlgren C F. Usefulness of Rajka & Langeland Eczema Severity Score in clinical practice. Acta Derm Venereol 2016; 96:521-4.).

If the infant is diagnosed with AD, the EASI and POEM, described below, will be employed to assess its severity at the time of diagnosis and at Weeks 12, 52, and 104.

Eczema Area and Severity Index (EASI)<8 years of age:

The EASI is a tool used to measure the extent (area) and severity of atopic eczema/AD (Hanifin J M, Thurston M, Omoto M, Cherill R, Tofte S J, Graeber M. The eczema area and severity index (EASI): assessment of reliability in atopic dermatitis. EASI Evaluator Group. Exp Dermatol 2001; 10:11-8.). The instrument assesses four body regions: the head and neck (including face, neck, and scalp), trunk (including genital area), upper limbs (including hands), and lower limbs (including buttocks and feet), which are assigned proportionate body surface areas of 20%, 30%, 20%, and 30%, respectively. An area score is determined for each of these four body regions based on the percentage of skin affected by AD (as defined by the four key signs listed below) within that body region (0=None, 1=1-9%, 2=10-29%, 3=30-49%, 4=50-69%, 5=70-89%, 6=90-100%). Each of the four body regions is also assessed for the severity of four key signs of AD—erythema, induration/papulation/edema, excoriation, and lichenification—using a 0 to 3 scale: 0=none; 1=mild; 2=moderate; and 3=severe (note: half-points are allowed). The total score for each body region is determined by multiplying the sum of the severity scores of the four key signs by the area score, then multiplying the result by the constant body surface area assigned to that body region. The total EASI score is the sum of the body region scores and ranges from 0 to 72. A trained physician designated will assess AD severity using the EASI at time of AD diagnosis and (only for subjects with AD) at Weeks 12, 52, and 104.

### Patient-Oriented Eczema Measure (POEM):

The POEM is a simple, valid, easily interpreted, and reproducible tool for assessing AD and monitoring aspects of the disease that are important to patients (Charman C R, Venn A J, Williams H C. The Patient-Oriented Eczema Measure: Development and initial validation of a new tool for measuring atopic eczema severity from the patients' perspective. Arch Dermatol 2004; 140:1513-9.; Charman C R, Venn A J, Ravenscroft J C, Williams H C. Translating Patient-Oriented Eczema Measure (POEM) scores into clinical practice by suggesting severity strata derived using anchor-based methods. Br J Dermatol 2013; 169:1326-32.). Study Personnel will interview Caregivers at time of AD diagnosis and (only for subjects with AD) at Weeks 12, 52, and 104 to rate seven symptoms (itchy skin, sleep disturbance, bleeding skin, skin weeping/oozing, skin flaking, skin cracking, skin dryness/roughness) using a 5-point scale of frequency of occurrence during the previous week (no days, 1-2 days, 3-4 days, 5-6 days, every day). The maximum total POEM score is 28.

### Infantile Colic

Study Personnel will interview Caregivers to document signs and symptoms of infantile colic at Baseline (Day 0) and at Weeks 6, 12, and 24 based on the Rome IV criteria (Benninga M, Nurko S, Faure C, Hyman P, St. James-Roberts I, Schechter N. Childhood functional gastrointestinal disorders: neonate/toddler. Gastroenterology 2016; 150:1443-55). As defined by the Rome IV criteria, the occurrence of infantile colic will be determined utilizing these Caregiver interviews in combination with daily Caregiver entries in a diary. Caregivers will utilize the diary to document the occurrence and duration of crying, fussing, and other related symptoms such as bowel movements at Baseline and daily during Weeks 5-7, 11-13, and 23-24. “Fussing” will refer to intermittent distressed vocalization and has been defined as “[behavior] that is not quite crying but not awake and content either” (Benninga, 2016; Zeevenhooven J, Koppen I J, Benninga M A. The new Rome IV criteria for functional gastrointestinal disorders in infants and toddlers. Pediatr Gastroenterol Hepatol Nutr 2017; 20:1-13). As defined by the Rome IV criteria, the occurrence of infantile colic will be determined utilizing Caregiver diary entries in combination with the Caregiver interviews conducted at the visits.

### Infant Anthropometrics

Infants will have their length/height (using an infant length board), body weight, and head circumference measured during all study visits. The Body Mass Index (BMI) will be calculated based on measurements of body weight and body length/height according to the following equation: BMI (kg/m²)=weight (kg)/([length or height in cm/100]²).

### Brief Infant Sleep Questionnaire—Revised (BISQ-R)

At Weeks 12, 24, 52, and 76, Caregivers will document the infant's sleep routines and patterns during the previous 2 weeks using the BISQ-R. The BISQ-R is an age-based, norm-referenced scoring system that provides a comprehensive assessment of infant and toddler sleep patterns (5 items related to sleep onset latency, number and duration of night wakings, longest stretch of sleep, and total night sleep), as well as parent perception (3 items related to bedtime difficulty, overnight sleep, overall child sleep problems) and parent behaviors (11 items related to bedtime routine consistency, bedtime, parental behavior at time of sleep onset and following night wakings, and sleep locations at time of sleep onset and following night wakings) that may impact sleep outcomes (Mindell J A, Gould R A, Tikotzy L, Leichman E S, Walters R M. Norm-referenced scoring system for the Brief Infant Sleep Questionnaire—Revised (BISQ-R). Sleep Med 2019; 63:106-14.). A score ranging from 0-100 is derived for each sub-scale, with higher scores denoting better sleep quality, more positive perceptions of sleep quality, and parental habits that promote healthy sleep behaviors and independent infant sleep, respectively. A total score is calculated as the average of the infant sleep, parent perceptions, and parent behavior subscale scores.

### Solid Food Introduction Checklist

Caregivers will document the first time the infant ingests a new solid food, to include the type and amount of the solid food, as applicable.

### Infant Feeding and Study Supplement Usage Log

Caregivers will record details of maternal breastfeeding, infant formula use (if applicable), and administration of the Study Supplement in a diary on a daily basis through Week 12 and optionally thereafter. Caregivers will also document any missed, incomplete, or extra administrations of the Study Supplement in the diary; missed supplementations will not be replaced on subsequent days.

### Blood Samples

A 2.0 mL venous whole blood sample will be collected from infants at Weeks 24, 52, and 104. The blood will be processed and archived according to instructions provided in the Laboratory Reference Manual for future analyses. Allergen-specific serum IgE tests will be conducted using the venous whole blood samples to evaluate the development of allergic sensitization. In addition, the blood samples may also be used to investigate the infant immune responder phenotype by comparing serial RNA expression profiles of subjects in the B. infantis (EVC001) supplementation group and in the placebo supplementation group. The blood samples may also be used for exome-sequencing to determine the presence of variations in different genes, including the FLG gene, that might relate to atopic disease (e.g. AD, asthma, and allergic sensitization) and ichthyosis vulgaris.

### Stool Samples

Maternal stool samples for future microbiome analysis will be collected 2-6 weeks postpartum, if the mother has consented to the collection (optional). Caregivers will collect infants' stool within 5 days (preferably within 3 days) prior to the Baseline (Day 0) and Weeks 6, 12, 24, 52, and 104 visits. The Baseline sample should be a non-meconium sample; if this is not possible prior to the start of supplementation, a deviation should be recorded and a non-meconium sample should be collected as soon as possible once supplementation has begun (preferably within 24 hours).

All stool samples will be collected using the provided supplies and stored frozen within a provided collection tube and biohazard bag until collected by Study Personnel during an in-person visit. Soft-sided coolers fitted with ice packs will be utilized to transport frozen stool samples to the Study Site where they will be stored at −80° C. for future analyses.

Stool collected at Baseline (Day 0) and Weeks 6, 12, 24, 52, and 104 will be analyzed for the overall bacterial profile of the gut including the presence of B. infantis to determine the extent (percentage of B. infantis colonization within each infant) and incidence (percentage of infants with gut B. infantis colonization) of colonization. An infant's gut will be considered colonized if the fecal concentration of B. infantis, determined by shotgun sequencing, is ≥50% of the total bacteria.

### Skin Swab Samples

Caregivers will be instructed to avoid applying any topical treatment within 3 hours prior to a study visit so as not to interfere with collection of skin samples. All skin samples, collected as detailed below, will be stored frozen at −80° C. at the Study Site for future analysis, as detailed in the Laboratory Reference Manual.

Skin biomarkers: To assess the effects of B. infantis (EVC001) supplementation on immune regulation in the skin and to learn about biomarkers associated with the onset of AD, Study Personnel will collect two skin samples using FibroTX Skin Sample Collection Swabs at Baseline (Day 0), Week 12, and Week 52; if AD is diagnosed, two additional samples will be collected from lesional and adjacent clear nonlesional locations at these timepoints. The FibroTX Skin Sample Collection Swab is a highly sensitive multi-analyte research tool for noninvasive biomarker measurements directly from skin.

Skin microbiome: To assess the systemic effects of the Study Supplement on the skin's microbiome, Study Personnel will utilize pre-moistened swabs to collect two skin samples at Baseline (Day 0), Week 6, Week 12, and Week 52, one from the antecubital fossa (elbow crease) and the other at a location determined by Study Personnel or Sponsor. If AD is confirmed, two additional samples will be obtained from lesional skin and adjacent clear non-lesional skin at these timepoints.

### Study Visits and Evaluations:

A summary of the study procedures and evaluation schedule is shown in below in Table 2.

TABLE 2
Schedule of Events

| Study Period | | | | |
|---|---|---|---|---|
| | | | Early | Unscheduled |
| Screening | Supplementation Period | Follow-up Period | D/C | Study Visit |

| Study Visit^{a, b} | | | | | | | |
|---|---|---|---|---|---|---|---|
| Within | | | | | | | |
| 14 days | | | Week 12 | | Week 52 | | Week 104 |
| postpartum^{c} | Day 0 | Week 6 | (~3 months) | Week 24 | (~1 year) | Week 76 | (~2 years) |

Additional definitions

| | | |
|---|---|---|
| Baseline^{d} | EOS | D/C^{e} |

Administrative/Eligibility

| | | | | | | |
|---|---|---|---|---|---|---|
| ICD (and sub-study | X | | | | | |
| addendum, if | | | | | | |
| applicable) | | | | | | |
| (see Section 4.1) | | | | | | |
| Demographic | X | | | | | |
| Questionnaire | | | | | | |
| Medical, Family, | X | | | | | |
| Medication, and | | | | | | |
| Supplement History | | | | | | |
| Questionnaire | | | | | | |
| initial inclusion/ | X | | | | | |
| exclusion criteria | | | | | | |
| review^{f} | | | | | | |
| Final inclusion/ | | X | | | | |
| exclusion | | | | | | |
| criteria review | | | | | | |
| Caregiver provided | X | X | X | X | X | X |
| with instructions | | | | | | |
| and supplies for | | | | | | |
| subsequent stool | | | | | | |
| sample collection(s)^{g} | | | | | | |

Randomization and Study Supplement

| Enrollment/ | X | | |
|---|---|---|---|
| randomization | | | |
| Caregiver training on | X | | |
| Study Supplement | | | |
| administration and | | | |
| infant skin monitoring | | | |
| eDiary installation, | X | | |
| registration, and | | | |
| training | | | |
| Dispense Study | X | | |
| Supplement | | | |
| Administer Study | Once daily | | |
| Supplement | | | |
| Final Study Supplement | | X | X^{h} |
| accountability | | | |

Study Assessments/Ongoing Review Conducted by Study Personnel

| Lactation support for | X | X | X | X | | | | | X^{j} |
|---|---|---|---|---|---|---|---|---|---|
| Caregiversⁱ | | | | | | | | | |
| Infant skin monitoring | X | X | X | X | X | X | X | X | X |
| (e.g., AD, skin rash, | | | | | | | | | |
| diaper rash, itch)^{k} | | | | | | | | | |
| Interview Caregivers and | | | | | | | | | |
| (as applicable) review | | | | | | | | | |
| medical records for: | | | | | | | | | |
| Infant AEs | X | X | X | X | X | X | X | X | X |
| Concurrent product use | X | X | X | X | X | X | X | X | X |
| Signs of atopic disease | X | X | X | X | X | X | X | X | X |
| other than AD^{l} | | | | | | | | | |
| (asthma, allergic rhinitis, | | | | | | | | | |
| food allergy) | | | | | | | | | |
| Signs and symptoms of | X | X | X | X | | | | X^{m} | |
| infantile colic (based on | | | | | | | | | |
| Rome IV criteria) | | | | | | | | | |
| Infant routines and care | X | X | X | X | X | X | X | X | |
| (e.g., daycare, bowel | | | | | | | | | |
| movements, updated | | | | | | | | | |
| allergen exposures) | | | | | | | | | |
| POEM (only for infants | At time of AD diagnosis and at Weeks 12, 52, and 104 | | | | | | | X | Xⁿ |
| with AD) | | | | | | | | | |
| EASI (only for infants | At time of AD diagnosis and at Weeks 12, 52, and 104 | | | | | | | X | Xⁿ |
| with AD) | | | | | | | | | |
| Infant anthropometrics^{o} | X | X | X | X | X | X | X | X | X |

Data Collected via eDiary^{p}

| Infant Feeding and Study | Daily | | | Optional completion of feeding log | | |
|---|---|---|---|---|---|---|
| Supplement Usage Log | | | | | | |
| Infantile colic (e.g., | X | X | X | X | | |
| crying/fussiness; based | | Weeks | Weeks | Week | | |
| on Rome IV criteria) | | 5-7 | 11-13 | 23-24 | | |
| BISQ-R | | | X | X | X | X |

| | |
|---|---|
| Solid Food Introduction | Complete when new food introduced (one time per food) |
| Checklist | |

Biological Sample Collection

| | | | | | | | |
|---|---|---|---|---|---|---|---|
| Blood samples: | | | | | | | |
| 2.0 mL venous sample | | | | X^{q} | X^{q} | X^{q} | X^{r} |
| Stool samples: | | | | | | | |
| Infant gut microbiome | X | X | X | X | X | X | X^{r} |
| (including B. infantis | | | | | | | |
| gut colonization)^{s} | | | | | | | |
| Maternal gut | | X | | | | | |
| microbiome^{s, t} | | | | | | | |
| Skin samples (swabs): | | | | | | | |
| Biomarker profile | X^{u} | | X^{u} | | X^{u} | | X^{r, u} |
| (FibroTX kit) | | | | | | | |
| Microbiome | X^{u} | X^{u} | X^{u} | | X^{u} | | X^{r, u} |

### Study Endpoints and Data Analysis

Primary Endpoint: Cumulative Incidence of AD through Week 52. The cumulative incidence of AD through Week 52 will be compared between supplementation groups.

### Secondary Endpoints:

Distributions for time to onset of AD through Week 104 will be compared between supplementation groups
Cumulative incidence of AD through Weeks 24 and 104
Proportion of Infants with B. infantis Gut Colonization at Week 12: Week 12 stool samples will be analyzed for B. infantis colonization and for the overall bacterium load to determine the proportion of infants with B. infantis gut colonization. An infant's gut will be considered colonized as defined above.
AD severity based on the EASI score at time of AD onset and at Weeks 12, 52, and 104
AD severity based on the POEM score at time of AD onset and at Weeks 12, 52, and 104

### Additional Endpoints:

Cumulative Incidences of Atopic Disease Other than AD through Weeks 24, 52, and 104: Study Personnel will document the development of allergic rhinitis, asthma, and food allergy (as confirmed by a specialist/pediatric allergist) throughout the study, and cumulative incidences of these disorders will be determined at Weeks 24, 52, and 104.
Cumulative Incidence of Allergic Sensitization through Weeks 24, 52, and 104: Study Personnel will document the development of allergic sensitization such as against dietary or inhaled allergens (as confirmed by specific serum IgE testing), and cumulative incidences will be determined at Weeks 24, 52, and 104.
Changes from Baseline in Gut and Skin Microbiome through Week 104: Changes from baseline in fecal (Weeks 6, 12, 24, 52, and 104) and skin (Weeks 6, 12, and 52) microbiome will be determined.
Proportion of Infants with B. infantis Gut Colonization at Week 24: Stool samples collected in association with the Week 24 visit will be analyzed for B. infantis gut colonization as detailed above.
Changes from Baseline in Skin Immunological Biomarker Profile at Weeks 12 and 52: Changes from baseline in skin biomarkers at Weeks 12 and 52 will be determined.
Cumulative Incidence of Infantile Colic through Weeks 6, 12, and 24: A diagnosis of infantile colic will be based on the Rome IV criteria, which include data generated by visit interviews and Caregiver diary.
BISQ-R Scores at Weeks 12, 24, 52, and 76: BISQ-R infant sleep, parent perceptions, and parent behavior subscale scores, as well as the total BISQ-R score at Weeks 12, 24, 52, and 76 will be determined.
Changes from Baseline in Infant Anthropometrics at Weeks 6, 12, 24, 52, 76, and 104: Changes from baseline in infant's length/height, body weight, head circumference, and BMI at Weeks 6, 12, 24, 52, 76, and 104 will be determined.
Influence of Maternal Gut Microbiome on Infant Gut Microbiome Development from Baseline to Two Years of Age: The microbiome of maternal stool samples collected 2-6 weeks postpartum will be assessed for correlations with development of the infant gut microbiome, including infant B. infantis gut colonization, at Baseline (Day 0) and Weeks 6, 12, 24, 52, and 104.
Microbiome and Immunological Profiles in Infants With or Without AD: Outcomes measures relating to microbiome and immunological profiles will be characterized in and compared between infants who do not develop AD through 1 year and infants who do in the B. infantis (EVC001) supplementation group and in the placebo supplementation group.
Serial RNA Expression Analysis at Weeks 24, 52, and 104: Outcome measures relating to serial RNA expression profiles will be characterized in and compared between subjects in the B. infantis (EVC001) supplementation group and in the placebo supplementation group at Weeks 24, 52, and 104 to investigate the infant immune responder phenotype.
Subject Genetic Analysis: Outcome measures relating to subject genetic profiles will be characterized in and compared between infants who do not develop AD through 1 year and infants who do in the B. infantis (EVC001) supplementation group to identify subjects who are likely to benefit from the intervention.

### Success Criteria

The success of the study will primarily be determined by a statistically significant effect of supplementation versus placebo on the cumulative incidence of AD through Week 52.

### SUMMARY

The above example constitutes a method comprising administering a composition comprising a Bifidobacterium (B. infantis) to a breastfed infant having an increased risk of developing an atopic disease. Atopic dermatitis, food allergy, allergic rhinitis and asthma will be monitored and evaluated for indications of prevention, delay and/or amelioration in the breastfed infants. Infantile colic, infant sleep and infant anthropometrics will also be monitored and evaluated.

What is claimed is:

1. A method of preventing, delaying or ameliorating atopic dermatitis in a breastfed infant, the method comprising:
a. administering a composition comprising an effective amount of a Bifidobacterium selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof to the breastfed infant.

2. The method of claim 1, wherein the Bifidobacterium is selected from the group consisting of B. longum, B. breve, B. kashiwanohense and combinations thereof.

3. The method of claim 1 or 2, wherein the Bifidobacterium is a B. longum subspecies selected from the group consisting of longum, suis, infantis, and combinations thereof.

4. The method of any of claims 1-3, wherein the Bifidobacterium is B. infantis.

5. The method of any of claims 1-4, wherein the Bifidobacterium is mixed into breastmilk prior to administering the Bifidobacterium to the breastfed infant.

6. The method of any of claims 1-5, wherein the Bifidobacterium is mixed into infant formula prior to administering the Bifidobacterium to the breastfed infant.

7. The method of any of claims 1-6, wherein the Bifidobacterium is mixed with about 3 to about 5 mL of breastmilk, infant formula or water prior to administering the Bifidobacterium to the breastfed infant.

8. The method of any of claims 1-7, wherein the breastfed infant is greater than 50% breastfed.

9. The method of any of claims 1-8, wherein the breastfed infant is exclusively breastfed.

10. The method of any of claims 1-9, wherein the Bifidobacterium is in powder form mixed with lactose.

11. The method of any of claims 1-10, wherein about 5 to about 15 billion CFU of the Bifidobacterium are administered to the breastfed infant.

12. The method of any of claims 1-11, wherein the B. infantis is administered once daily.

13. The method of any of claims 1-12, wherein the B. infantis is first administered within the first 2 weeks of life.

14. The method of any of claims 1-13, wherein the B. infantis is administered before the first 12 weeks of life.

15. The method of any of claims 1-14, wherein the Bifidobacterium is first administered within the first 2 weeks and until the 12^{th} week of life.

16. The method of any of claims 1-15, wherein the breastfed infant has an increased risk of developing an atopic disease.

17. The method of any of claims 1-16, where in the Bifidobacterium comprises the strain EVC001.

18. A method of preventing, delaying, or ameliorating atopic dermatitis in a breastfed infant having at least one first-degree relative with history of an atopic disease, the method comprising:
a. administering once daily an effective amount of B. infantis mixed with breastmilk to the breastfed infant,
wherein the breastfed infant is at least 90% breastfed, and wherein the B. infantis is first administered within first 2 weeks of life.

19. The method of claim 18, wherein the B. infantis comprises strain EVC001.

20. The method of claim 18, or 19 wherein the B. infantis is mixed with about 3 to about 5 mL of breastmilk or infant formula prior to administering the B. infantis to the breastfed infant.

21. The method of any of claims 18-20, wherein the breastfed infant is exclusively breastfed.

22. The method of any of claims 18-21, wherein the B. infantis is in powder form mixed with lactose.

23. The method of any of claims 18-22, wherein 8 billion CFU of B. infantis are administered to the breastfed infant.

24. The method of any of claims 18-23, wherein the B. infantis is administered once daily until the 12^{th} week of life.

25. A method of preventing, delaying or ameliorating an atopic disease selected from the group consisting of food allergy, allergic rhinitis, asthma and combinations thereof in a breastfed infant, the method comprising administering a composition comprising an effective amount of a Bifidobacterium to the infant.

26. The method of claim 25, wherein the Bifidobacterium is selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof to the breastfed infant.

27. The method of claim 25 or 26, wherein the Bifidobacterium is selected from the group consisting of B. longum, B. breve, B. kashiwanohense and combinations thereof.

28. The method of any of claims 25-27, wherein the Bifidobacterium is a B. longum subspecies selected from the group consisting of longum, suis, infantis, and combinations thereof.

29. The method of any of claims 25-28, wherein the Bifidobacterium is B. infantis.

30. The method of any of claims 25-29, wherein the Bifidobacterium is mixed into breastmilk prior to administering the Bifidobacterium to the breastfed infant.

31. The method of any of claims 25-30, wherein the Bifidobacterium is mixed into infant formula prior to administering the Bifidobacterium to the breastfed infant.

32. The method of any of claims 25-31, wherein the Bifidobacterium is mixed with about 3 to about 5 mL of breastmilk, infant formula, or water prior to administering the Bifidobacterium to the breastfed infant.

33. The method of any of claims 25-32, wherein the breastfed infant is greater than 50% breastfed.

34. The method of any of claims 25-33, wherein the breastfed infant is exclusively breastfed.

35. The method of any of claims 25-34, wherein the Bifidobacterium is in powder form mixed with lactose.

36. The method of any of claims 25-35, wherein about 5 to about 15 billion CFU of the Bifidobacterium are administered to the breastfed infant.

37. The method of any of claims 25-36, wherein the B. infantis is administered once daily.

38. The method of any of claims 25-37, wherein the B. infantis is first administered within the first 2 weeks of life.

39. The method of any of claims 25-38, wherein the B. infantis is administered before the first 12 weeks of life.

40. The method of any of claims 25-39, wherein the Bifidobacterium is first administered within the first 2 weeks and until the 12^{th} week of life.

41. The method of any of claims 25-40, wherein the breastfed infant has an increased risk of developing the atopic disease.

42. The method of any of claims 25-41, where in the Bifidobacterium comprises the strain EVC001.

43. A method of improving infantile colic, infant sleep or infant anthropometrics in a breastfed infant, the method comprising administering a composition comprising an effective amount of a Bifidobacterium to the infant.

44. The method of claim 43, wherein the Bifidobacterium is selected from the group consisting of B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense A, B. italicum, B. imperatoris, B. cricetid, B. catulorum, B. callitrichidarum, B. animalis, B. aesculapii, and combinations thereof to the breastfed infant.

45. The method of claim 43 or 44, wherein the Bifidobacterium is selected from the group consisting of B. longum, B. breve, B. kashiwanohense and combinations thereof.

46. The method of claim 45, wherein the Bifidobacterium is a B. longum subspecies selected from the group consisting of longum, suis, infantis, and combinations thereof.

47. The method of any of claims 43-46, wherein the Bifidobacterium is B. infantis.

48. The method of any of claims 43-47, wherein the Bifidobacterium is mixed into breastmilk prior to administering the Bifidobacterium to the breastfed infant.

49. The method of any of claims 43-48, wherein the Bifidobacterium is mixed into infant formula prior to administering the Bifidobacterium to the breastfed infant.

50. The method of any of claims 43-49, wherein the Bifidobacterium is mixed with about 3 to about 5 mL of breastmilk, infant formula, or water prior to administering the Bifidobacterium to the breastfed infant.

51. The method of any of claims 43-50, wherein the breastfed infant is greater than 50% breastfed.

52. The method of any of claims 43-51, wherein the breastfed infant is exclusively breastfed.

53. The method of any of claims 43-52, wherein the Bifidobacterium is in powder form mixed with lactose.

54. The method of any of claims 43-53, wherein about 5 to about 15 billion CFU of the Bifidobacterium are administered to the breastfed infant.

55. The method of any of claims 43-54, wherein the B. infantis is administered once daily.

56. The method of any of claims 43-55, wherein the B. infantis is first administered within the first 2 weeks of life.

57. The method of any of claims 43-56, wherein the B. infantis is administered before the first 12 weeks of life.

58. The method of any of claims 43-57, wherein the Bifidobacterium is first administered within the first 2 weeks and until the 12^{th} week of life.

59. The method of any of claims 43-58, wherein the breastfed infant has an increased risk of developing the atopic disease.

60. The method of any of claims 43-59, where in the Bifidobacterium comprises the strain EVC001.
